(19) United States
(12) Patent Application Publication
(10) Pub. No.: US 2023/0184761 A1
(43) Pub. Date: Jun. 15, 2023

(54) METHOD OF DETECTING TB IN BLOOD SAMPLE

(71) Applicant: The Administrators of the Tulane Educational Fund, New Orleans, LA (US)

(72) Inventors: Ye Tony HU, New Orleans, LA (US); Wenshu ZHENG, New Orleans, LA (US)

(21) Appl. No.: 18/164,679

(22) Filed: Feb. 6, 2023

Related U.S. Application Data
(63) Continuation of application No. PCT/US21/45086, filed on Aug. 6, 2021.
(60) Provisional application No. 63/061,674, filed on Aug. 5, 2020.

(51) Int. Cl. G01N 33/569; G01N 33/68; G01N 33/543

(52) U.S. Cl. CPC G01N 33/5695; G01N 33/6893; G01N 33/54346; G01N 2469/10

(57) ABSTRACT

A method and system for detecting TB in a bodily fluid sample are described. By extracting extracellular vesicles (EVs) from the bodily fluid sample and employing antibody-conjugated nanoparticles to capture Mtb-related biomarkers, it is shown that the test can be completed within hours instead of weeks, and the detection limit can be significantly lowered with high accuracy. Additionally, the method and system described is capable of distinguishing between active and latent TB infections.

### PRIOR RELATED APPLICATIONS

This application claims priority to U.S. Provisional Application No. 63/061,674, filed Aug. 5, 2020, which is incorporated by reference in its entirety for all purposes.

### FEDERALLY SPONSORED RESEARCH STATEMENT

Not applicable.

### FIELD OF THE DISCLOSURE

The disclosure generally relates to a method for diagnosis tuberculosis in a bodily fluid sample, and more particularly relates to a method for detecting tuberculosis-derived antigen on circulating extracellular vesicles (EVs) in the bodily fluid sample by nano plasmon enhancement assay using dark field microscopy.

### BACKGROUND OF THE DISCLOSURE

Global tuberculosis becoming a more severe burden than early estimation, as potentially millions are neither diagnosed nor reported each year. Timely diagnosis of tuberculosis disease remains urgent for positive patient outcomes. However, traditional techniques such as Mycobacterium tuberculosis (MTB) culture and sputum smear are time-consuming and suffer low sensitivity. Xpert MTB/RIF assay enables the rapid diagnosis of TB as well as the assessment of rifampicin resistance within 2 hours, while the sensitivity of this assay relies on a high bacterial load on sputum samples. Despite tremendous efforts in exploring pathogen-derived antigenic markers for TB. Only lipoarabinomannan (LAM), a TB antigen that is identified to shed into the urine during active TB, was reviewed by WHO to enable rapid early screen of HIV positive TB patients. However, Failure to detect LAM in the urine of HIV-negative patients limits the applicability of these assays, as 85% of the TB patients are HIV-negative. In addition, this assay can be only applied for urine samples.

An ideal invasive test should measure both pathogenetic and host targets in body fluids to achieve the highest accuracy for TB at different phase, including latent infection. Extracellular vesicles (EVs) that are heavily implicated in pathogenic process could contain both many host and constituents for maker discovery. As a widely studied and well-established TB specific maker, the failure for the detection of LAM in HIV-negative patients could be due to that LAM is masked by formation of immune complexes. LAM is well confirmed to mediate the release form EVs form macrophage.

Therefore, there is a need to develop an assay to enable rapid, quantitative, ultrasensitive, non-sputum-based biomarker in detecting active TB.

### SUMMARY OF THE DISCLOSURE

In one aspect of this disclosure, a method of detecting a Mycobacterium tuberculosis (MTB)-specific protein in a bodily fluid sample is described. The method comprises the steps of: (a) extracting extracellular vesicles (EVs) in the bodily fluid sample; (b) mixing antibody-conjugated nanoparticles with the EVs in step (a), wherein the antibody-conjugated nanoparticles are conjugated with a first antibody against the MTB-specific protein; and (c) detecting the presence of the MTB-specific protein using dark field microscopy.

In another aspect of this disclosure, a system for detecting an MTB-specific protein in a bodily fluid sample is described. The system comprises: (a) a dark field microscope; (b) a sample substrate; and (c) antibody-conjugated nanoparticles, wherein said antibody-conjugated nanoparticles are conjugated with a first antibody that targets the MTB-specific protein; wherein the sample substrate is coated with a second antibody against an extracellular vesicle-specific (EV-specific) protein.

In another aspect of this disclosure, a method of detecting and determining tuberculosis infection status by detecting the presence of a first and a second Mycobacterium tuberculosis (MTB)-specific proteins in a bodily fluid sample is described. The method comprises the steps of: a) extracting extracellular vesicles (EVs) in the bodily fluid sample; b) mixing antibody-conjugated nanoparticles with the extracted EVs in step a), wherein said antibody-conjugated nanoparticles are conjugated with a first antibody against the first MTB-specific protein and a second antibody against the second MTB-specific protein; c) detecting the presence of the first and/or the second MTB-specific proteins using dark field microscopy; and d) determining the tuberculosis infection status based on the presence of the first and the second MTB-specific proteins.

In another aspect of this disclosure, a method of screening antibodies against an MTB-specific protein is described. The method comprises the steps of: (a) immobilizing an MTB-specific protein on a substrate; (b) introducing a plurality of first antibody onto the substrate; (c) mixing nanoparticles with the mixture in step (b), wherein the nanoparticles are conjugated with a second antibody and signal-emitting groups, wherein the second antibody targets the heavy chain constant region of the first antibody; and (d) detecting the presence of antibodies against the MTB-specific protein by detecting the signals emitted by the signal-emitting groups on said nanoparticles.

In another aspect of this disclosure, a method of detecting the presence of a bacterium-specific protein in a bodily fluid sample is described. The method comprises: (a) extracting extracellular vesicles (EVs) in the bodily fluid sample; (b) mixing antibody-conjugated nanoparticles with the EVs, wherein said antibody-conjugated nanoparticles are conjugated with a first antibody specific to said bacterium-specific protein; and (c) detecting the presence of the bacterium-specific protein using dark field microscopy.

In one embodiment, the bodily fluid sample can be obtained from a patient. For example, the bodily fluid can be blood, serum, sputum, urine, or other available bodily fluid.

In one embodiment, the antigen of the first antibody is selected from the group of antigens specific to Mtb, and the group of Mtb-specific antigens is consisted of lipoarabinomannan (LAM), Antigen 85B, LAM carrier protein LprG and LpqH, Alpha-crystallin (HspX), DnaK, GroEL2, KatG, SodA and GlnA. In one embodiment, the antigen of the second antibody is selected from the same group but different from the first antibody.

In one embodiment, the EVs are extracted by using a capture antibody such as anti-CD81 or anti-MTB antibodies. Detection antibodies that recognizes surface markers on exosome or MTB-derived EVs can also be used, and non-limiting examples include CD9, CD91, CD63, PDCD6IP, HSPA8, ACTB, ANXA2, PKM, HSP90AA1, ENO1, ANXAS, HSP90AB1, YWHAZ, YWHAE, LprG, LpqH, Alpha-crystallin (HspX), DnaK, GroEL2, KatG, SodA and GlnA etc. In one embodiment, an anti-CD81 antibody is used to extract the EVs.

In one embodiment, the nanoparticles are those capable of generating a surface plasmon resonance effect when excited by a light source to substantially increase the fluorescence emitted by the inorganic fluorescent particle. Gold nanoparticles are most commonly used due to its stability and easily modifiable surface. To conjugate with antibodies, the surface of the gold nanoparticles can be modified, for example, with carboxyl group. The negatively charged gold nanoparticles can therefore form covalent bonds with positively charged amine groups. However, other nanoparticles may also be used, and non-limiting examples include nanoparticles made of silver (Ag), bismuth oxide (Bi₂O₃), platinum (Pt), gadolinium oxide (Gd₂O₃), iron oxide (Fe₃O₄), etc., as long as these particles can show an enhanced scattering light.

In another aspect of this disclosure, a system for screening antibodies against an MTB-specific protein is described. The system comprises a substrate, wherein the substrate has a coating that forms covalent bonding with the MTB-specific protein; the MTB-specific protein; and nanoparticles conjugated with an antibody against heavy chain constant region of an IgG.

As used herein, “Mycobacterium tuberculosis” or “MTB” refers to a species of pathogenic bacteria in the family Mycobacteriaceae and the causative agent of tuberculosis. MTB has a slow growth rate, with doubling time approximately once per day.

As used herein, “sample” refers to a small amount of biological substance collected from a person to be examined.

As used herein, “sample substrate” refers to a substrate onto which a sample may sit and be examined by the microscopy. In one embodiment, the sample substrate is a glass slide, such as a patterned glass slide on which multiple samples may be deposited. However, other sample substrates can also be used, for example, slides or transparent plates made of other materials.

As used herein, “extracellular vesicles” or “EV” refers to lipid bilayer-delimited particles that are naturally released from a cell, bacterial and cannot replicate themselves. EVs range in diameter from about 20-30 nm to about 10 μm or more. EVs are capable of transferring nucleic acids, such as RNA, between cells.

EVs are typically separated from a bodily fluid sample by ultracentrifuge or density gradient ultracentrifugation, size exclusion chromatography, ultrafiltration, and affinity/immunoaffinity capture method. There are certain EV-enriched markers that can be used to better isolate EVs. Examples of EV-enriched markers include, but not limited to, CD81, PDCD6IP, HSPA8, ACTB, ANXA2, CD9, PKM, HSP90AA1, ENO1, ANXA5, HSP90AB1, CD63, YWHAZ, YWHAE, etc. as well as the antibody against LprG, LpqH, Alpha-crystallin (HspX), DnaK, GroEL2, KatG, SodA and GlnA.

As used herein, “dark field microscopy” refers to microscopy methods that exclude the un-scattered beam from the image such that the background around the specimen is dark. In optical microscopy, dark-field describes an illumination technique used to enhance the contrast in unstained samples by illuminating the sample with light that will not be collected by the objective lens and thus will not form part of the image. When coupled to hyperspectral imaging, dark-field microscopy can be used to characterize the nanomaterials embedded in cells, such as gold nanoparticles targeting cells with certain markers.

As used here, “antibody-conjugated nanoparticles” refers to nanoparticles that are conjugated with specific antibodies against a target antigen. The conjugation between the antibodies and the nanoparticles can be electrostatic interaction (physical adsorption) or covalent conjugation in the orientation of antibodies on the metallic surface as coupling method. Static, ionic adsorption of antibodies with nanoparticles have been reported to have poor reproducibility, random orientation of the antibodies, and low stability at different pH conditions. Covalent coupling by modifying the surface of nanoparticles with reactive groups (such as carboxyl and amine groups) facilitates bioconjugation with the amino acid side chains of the antibody.

As used here, Antigen 85B refers to a subunit of antigen 85 (Ag85) complex (Ag85A, Ag85B, Ag85C) that is found to be produced in MTB culture fluid. The 85A, 85B and 85C proteins are encoded by three genes located at different sites in the mycobacterial genome and show extensive cross-reactivity as well as homology at amino acid and gene level.

As used herein, “LAM carrier protein LprG” refers to Lipoarabinomannan carrier protein LprG.

As used herein, “LpqH” refers to Lipoprotein LpqH found in MTBs. The 19 kDa Mycobacterium tuberculosis lipoprotein (LpqH) induces macrophage apoptosis through extrinsic and intrinsic pathways: a role for the mitochondrial apoptosis-inducing factor.

As used herein, “alpha-crystallin (HspX)” refers to a 16 kDa heat shock protein HspX that is required for mycobacterium persistence within microphages.

As used herein, “DnaK” refers to bacterial molecular Chaperone protein DnaK. Chaperones are proteins that bind to other proteins, thereby stabilizing them in an ATP-dependent manner. DnaK is an enzyme that couples cycles of ATP binding, hydrolysis, and ADP release by an N-terminal ATP-hydrolysing domain to cycles of sequestration and release of unfolded proteins by a C-terminal substrate binding domain.

As used herein, “GroEL2” refers to the 60 kDa chaperonin 2 (aka Cpn60.2) that is closely related to Cpn60.1 chaperone localized within the outer layer of M. tuberculosis cell wall. GroEL2 is found to be present in the cerebrospinal fluid of TB meningitis patients.

As used herein, “KatG” refers to Catalase-peroxidase, which activates the pro-drug INH that is coded by the katG gene in M. tuberculosis. Mutations of the katG gene in M. tuberculosis are a major INH resistance mechanism.

As used herein, “SodA” refers to Superoxide dismutase [Fe]. For MTB detection purposes, unless otherwise specified, SodA refers particularly to MTB SodA.

As used herein, “GlnA” refers to Glutamine synthetase. For MTB detection purposes, GlnA refers particularly to MTB GlnA.

As used herein, “PDCD6IP” refers to programmed cell death 6-interacting protein, which encodes a protein thought to participate in programmed cell death.

As used herein, “HSPA8” refers to human heat shock 70 kDa protein 8, also known as heat shock cognate 71 kDa protein or Hsc70 or Hsp73. As a member of the heat shock protein 70 family and a chaperone protein, it facilitates the proper folding of newly translated and misfolded proteins, as well as stabilizing or degrading mutant proteins.

As used herein, “ACTB” refers to human beta-actin, which is one of six different actin isoforms that have been identified in humans.

As used herein, “ANXA2” refers to annexin A2, which is involved in diverse cellular processes such as cell motility, linkage of membrane-associated protein complexes to the actin cytoskeleton, endocytosis, fibrinolysis, ion channel formation, and cell matrix interaction.

As used herein, “PKM” refers to pyruvate kinase M1/2, which catalyzes the transfer of a phosphoryl group from phosphoenolpyruvate to ADP, generating ATP and pyruvate.

As used herein, “HSP90AA1” refers to human heat shock protein HSP 90-alpha (cytosolic), member Al Complemented by the constitutively expressed paralog Hsp90B which shares over 85% amino acid sequence identity, Hsp90A expression is initiated when a cell experiences proteotoxic stress. Once expressed Hsp90A dimers operate as molecular chaperones that bind and fold other proteins into their functional 3-dimensional structures.

As used herein, “ENO1” refers to alpha-enolase, which is a glycolytic enzyme expressed in most tissues. Each isoenzyme is a homodimer composed of 2 alpha, 2 gamma, or 2 beta subunits, and functions as a glycolytic enzyme. Alpha-enolase, in addition, functions as a structural lens protein (tau-crystallin) in the monomeric form.

As used herein, “ANXA5” refers to annexin A5, which is a cellular protein in the annexin group. ANXA5 is able to bind to phosphatidylserine, a marker of apoptosis when it is on the outer leaflet of the plasma membrane.

As used herein, “HSP90AB1” refers to heat shock protein HSP 90-beta, a molecular chaperone.

As used herein, “YWHAZ” refers to 14-3-3 protein zeta/delta, which is a member of the 14-3-3 protein family and a central hub protein for many signal transduction pathways. It is a major regulator of apoptotic pathways critical to cell survival and plays a key role in a number of cancers and neurodegenerative diseases.

As used herein, “YWHAE” refers to 14-3-3-protein epsilon, a member of the 14-3-3 family that mediate signal transduction by binding to phosphoserine-containing proteins.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention.

The following abbreviations are used herein:

| ABBREVIATION | TERM |
|---|---|
| Ag85b | Antigen 85B |
| AuNRs | Gold Nanorods |
| EVs | Extracellular Vesicles or exosomes |
| LAM | lipoarabinomannan |
| LprG | Lipoarabinomannan carrier protein LprG |
| LprH | Putative lipoprotein LprH |
| LC | Liquid chromatography |
| MS | mass spectrometry |
| MTB | Mycobacterium tuberculosis |
| NEI | Nanoparticle-enhanced EV immunoassay |
| nPES | Nanoplasmon-enhanced scattering |
| TB | Tuberculosis |

### BRIEF DESCRIPTION OF THE DRAWINGS

FIG. 1. Purification and Characterization of EVs form TB-infected macrophage for marker discovery, including protein identification and lipoglycan confirmation. (a) Scheme of the in vitro TB infection model for marker discovery. (b) Numbers of different type of proteins identified from EVs that isolated from TB-infected macrophages using LC-MS proteomics. (c) Heatmap show the fold change of the several proteins between the culture medium and EVs that purified from TB-infected macrophages using LC-MS proteomics. (d) Western blot for the confirmation of LAM (37 kDa) from culture filter protein (CFP) from different TB stains, EVs and cytoplasm from TB infected macrophages.

FIG. 2. Nano plasmonic enhanced immunoassay for highly sensitive detection of TB antigen on TB-infected macrophages-derived EVs. (a) Scheme of TB antigen detection on EVs using AuNRs-based nano plasmonic enhanced immunoassay, (b) UV-vis spectrum of AuNRs. (c) TEM images of AuNRs. (d-g) Dark-field images of different concentration of AuNRs. (h) Quantification of the signal from the dark field images of different concentration of AuNRs. (i) Intensity quantified from dark field image of samples containing different concentration of LAM. means±SD; n=6. (j) Intensity of dark field image of samples containing 10 μg/mL TB-infected macrophages-derived EVs to the intensity in the absence of EVs using different combinations of capture antibody and detection antibody. (k) Intensity quantified from dark field image of samples containing different concentration of TB-infected macrophages-derived EVs using anti-CD81 antibody as the capture antibody and the anti-LAM antibody (A194-01) as the detection antibody, means±SD; n=6.

FIG. 3. The performance of TB diagnosis for pediatric, HIV negative patients using nano plasmonic enhanced immunoassay. (a) Intensity from the serum, purified EVs or supernatant after EVs purification from 4 TB patient serum samples, 2 control serum samples. (b) Intensity tested with nano plasmonic enhanced immunoassay and OD450 tested with ELISA from 15 TB patient serum samples, 5 control serum samples using anti-LAM as the detection antibody. (c) Intensity from TB patient samples and control samples when using anti-LAM antibody as the detection antibody using traditional ELISA, (d-f) Intensity from TB patient samples and control samples when using (d) anti-LAM, (e) anti-LprG, (f) anti-LpqH as the detection antibody. t-test, **, p<0.01, ***, p<0.001. (g) ROC curve for the diagnosis of 20 Vietnam samples using nano plasmonic enhanced immunoassay with different detection antibodies and ELISA using anti-LAM antibody.

FIG. 4A shows the rationale and assay schematic. OMVs: Mtb outer membrane vesicles.

FIG. 4B. Size distribution of EVs secreted by uninfected and Mtb H37Rv-infected macrophages.

FIG. 4C shows LAM and LprG on serum EVs of non-human primates with pulmonary TB (PTB), latent TB infection (LTBI), or their healthy controls (Ctrl) (Mean±SD of three technical replicates).

FIG. 4D shows EV-ELISA for LAM, LprG and integrated LAM and LprG (LAM+LprG) expression on serum EVs of children with TB (N=10) and without evidence of TB (Ctrl; N=5; Mean ±SE, *p<0.05 and **p<0.01 by non-parametric Kruskal-Wallis one-way ANOVA with Dunn's post-test).

FIG. 5A is a schematic of the NEI image capture workflow and signal.

FIG. 5B. EV LAM and LprG NEI signal linearity with an Mtb EV concentration curve generated using EVs from Mtb-infected macrophages.

FIG. 5C. Receiver operating characteristic (ROC) analysis for the ability of single and integrated EV LAM and LprG NEI signal to distinguish children with and without TB, indicating areas under the ROC curve.

FIG. 5D-F. NEI signal for (D) LAM, (E) LprG, and (F) integrated LAM and LprG expression on serum EVs of children with TB (N=10) and no evidence of TB (N=5). Solid lines indicate Mean±SE; dashed lines indicate the threshold for positive signal determined in corresponding ROC analysis (C)

FIG. 6. Mtb EV NEI diagnostic performance in children living with HIV at high risk of TB. NEI signal in children with confirmed, unconfirmed, and unlikely TB as determined by positive respiratory culture/Xpert or stool Xpert results, TB related symptoms meeting NIH criteria for the duration, chest X-ray (CXR) findings, close TB contact or positive TST, positive ATT response, and/or TB-related death.

FIG. 7A is a schematic of a portable smartphone-based DFM device for NEI assay readout.

FIG. 7B shows the darkfield condenser mask used in FIG. 7A.

### DETAILED DESCRIPTION

The disclosure provides novel method and system for detecting MTB presence in a bodily fluid sample and extracted EVs, as opposed to conventional TB testing method that requires sputum sample. Bodily fluid sample, such as blood, saliva or urine, is much easier to extract from a patient, whereas sputum sample is hard to obtain.

Further, the method and system of this disclosure can be used to quickly determine the presence of MTB within the matter of hours, even when the bodily fluid sample has only low amount of MTB proteins. Traditional TB testing method requires sputum culture, which would take 1-8 weeks. The fast turn-around timeframe helps doctors to make treatment decisions as well as preventing spread of TB.

Additionally, the method of this disclosure is capable of high accuracy detection of TB by extracting EVs without concentrating. In other practices, once the EVs are extracted, typically a centrifugation step is further performed to concentrate the EVs. In this disclosure, however, the highly sensitive nanoparticle and dark field microscope makes it much easier to detect TB even in bodily fluid samples without the need to further enrich the concentration of EVs in the sample.

Moreover, the method and system of this disclosure can be used to detect TB in non-HIV patients and latent patients. Conventional TB tests are only sensitive to HIV-positive patients, and also cannot distinguish latent TB patients from TB-negative patients. The method and system of this disclosure are shown to detect MTB presence in HIV-negative patients, and are also able to detect MTB presence in latent, asymptomatic TB patients. The high sensitivity and specificity of the method of system of this disclosure allows detection by using bodily fluid sample rather than sputum, as bodily fluid samples are much easier to obtain.

Perhaps more importantly, the method and system of this disclosure provides a combination of biomarkers that can be used to distinguish active TB patients from latent TB patients.

To achieve the results, the present disclosure describes a method for detecting the presence of MTB-specific proteins in a bodily fluid sample, comprising the steps of: a) extracting extracellular vesicles (EVs) in the bodily fluid sample; b) mixing nanoparticles with the EVs, wherein the nanoparticles are conjugated with a first antibody specific to the MTB-specific protein; and d) detecting the presence of MTB using dark field microscopy.

Alternatively, the present disclosure describes a method for screening antibodies for an MTB-specific protein, comprising the steps of: a) immobilizing an

MTB-specific protein on a substrate, b) introducing a plurality of first antibody onto the substrate; c) mixing nanoparticles with the mixture in step (b), wherein the nanoparticles are conjugated with a second antibody and signal-emitting groups, wherein the second antibody targets the heavy chain constant region of the first antibody; and d) detecting the presence of antibodies against the MTB-specific protein by detecting the signals emitted by the signal-emitting particles. This screening method can effectively screen a large number of antibodies to obtain the antibodies that target the MTB-specific protein.

The present disclosure also describes a system for detecting a MTB protein in a bodily fluid sample, comprising: a) a dark field microscope; b) a sample substrate; and c) antibody-conjugated nanoparticles, wherein the antibody-conjugated nanoparticles comprise an anti-MTB antibody that targets the MTB protein, wherein the sample substrate is coated with a second antibody against an extracellular vesicle-specific protein.

Alternatively, the present disclose describes a method of detecting and determining tuberculosis infection status by detecting the presence of a first and a second Mycobacterium tuberculosis (MTB)-specific proteins in a bodily fluid sample is described. The method comprises the steps of: a) extracting extracellular vesicles (EVs) in the bodily fluid sample; b) mixing antibody-conjugated nanoparticles with the extracted EVs in step a), wherein said antibody-conjugated nanoparticles are conjugated with a first antibody against the first MTB-specific protein and a second antibody against the second MTB-specific protein; c) detecting the presence of the first and/or the second MTB-specific proteins using dark field microscopy; and d) determining the tuberculosis infection status based on the presence of the first and the second MTB-specific proteins.

The method and system of the present disclosure focuses on extracellular vesicles that in a subject have at least one MTB protein. EVs have their specific surface markers that can be targeted by antibodies, whereas the at least one MTB protein also have epitopes targeted by antibodies. As such, one can simultaneously detect both pathogenetic and host targets in body fluids that contain EVs.

The present disclosure describes a novel method of detecting the presence of

MTB in a sample by first extracting the extracellular vesicles (EVs) in the sample, followed by detecting the MTB-specific markers from the EVs. To do this, the first step is to identify the MTB-specific markers that are present in EVs, and can therefore be captured.

### 1. Identifying TB-Specific Markers

To explore the TB specific marker from EVs, macrophage was infected with TB to enable the in vitro isolation of EVs for proteomic study with the following steps. As shown in Figure la, marker discovery is carried out with an in vitro TB-infection model. After infecting the macrophages with TB, the extracellular vesicles (EVs) secreted by the macrophages were purified. Additionally, both liquid chromatography mass spectrometry-based proteomics (LC-MS proteomic) study for the protein marker identification and immunoassay for the glycolipid confirmation were performed.

### Collecting Macrophage Culture Medium After Infection by Alive MTB

It is important to base experiments on TB-Infected macrophage in order to provide more accurate and practical results. The following materials are used:

Total samples needed: Culture medium from T175 Flasks (4× 10× 35-52.5 ml=1400-2100 mL), each flask contain MTB infected (MOI=10:1) activated THP-1 (2.5×107) [4 h infection]

Cell, bacteria and reagents: THP-1 ATCC® TIB202™, M.tb H37Rv (Ideally some other strains with different virulence), RPMI 1640 PS free medium, RPMI 1640 (FBS free, PS free) medium.

THP-1 cell culture (ATCC® TIB202™) Thaw the frozen cells in a 37° C. water bath. Thawing should be rapid (approximately 2 minutes).

1. Transfer the vial contents to a centrifuge tube containing 9.0 mL complete culture medium. And spin at approximately 125× g for 5 minutes.
2. Count the cell numbers and viability rate (no less than 95%). Resuspend cell pellet with the complete medium at the final concentration of 3×105 viable cells/ml.
3. Incubate the culture at 37° C. in a suitable incubator with 5% CO2.
4. Subculture when cell concentration reaches 8×105 cells/ml. Do not allow the cell concentration to exceed 1×106 cells/ml. Centrifuge the culture medium and re-suspend the cell pellet in the same way as mentioned above.
5. When the total number reach 2.5×108 cells, equally distributed the cells into ten T175 flasks (35-52.5 ml) (2.5×107 cells/flask) and add 500 ng/mL PMA to differentiate the THP-1 cells to macrophage for 24 h.
6. Check the morphology of the cells, if they were already stretched well, remove the culture medium gently, and wash the cells with 37° C. PBS three times. Then cells are ready for the infection.

### Infection of THP-1 by Alive MTh

i). MOI=10, Total de-clumped Mtb bacteria (M.tb H37Rv stocks)=2.5×109 will be needed for THP-1 infection.
ii). Resuspend the Mtb pellet in a small amount (5-10 mL) of RPMI 1640 culture medium with 10% FBS and de-clumped by a brief bath sonication and passed through syringe fitted with 27-gauge needle at least ten times.
iii). Add more RPMI 1640 PS free medium (PS=penicillin and streptomycin) with 10%

FBS in and mix it well. Then aliquot the mixture into 10 flasks of the macrophage. (20 mL RPMI PS free medium per flask)

iv). Infection for 4 h in culturing condition.
v). After the infection, remove the mixture and wash the cells gently with 37° C. PBS, 10 ml/time, 3 times or more (check under microscopy).
vi). Add RPMI 1640 (FBS free, PS free) to the flasks, incubate for 48 h.
vii). Collect the culture medium. Filtrate the medium using 0.45 um filters to remove the bacteria. Keep the medium in the −80° C. refrigerator and inoculation little medium 3-4 weeks (Mtb) to guarantee no live bacteria existed.

The supernatants form the aforementioned culture medium are collected for EVs isolation and then LC-MS for proteomic study with standard procedure. The isolation of EVs is well known in the field, and thus will not be repeated herein.

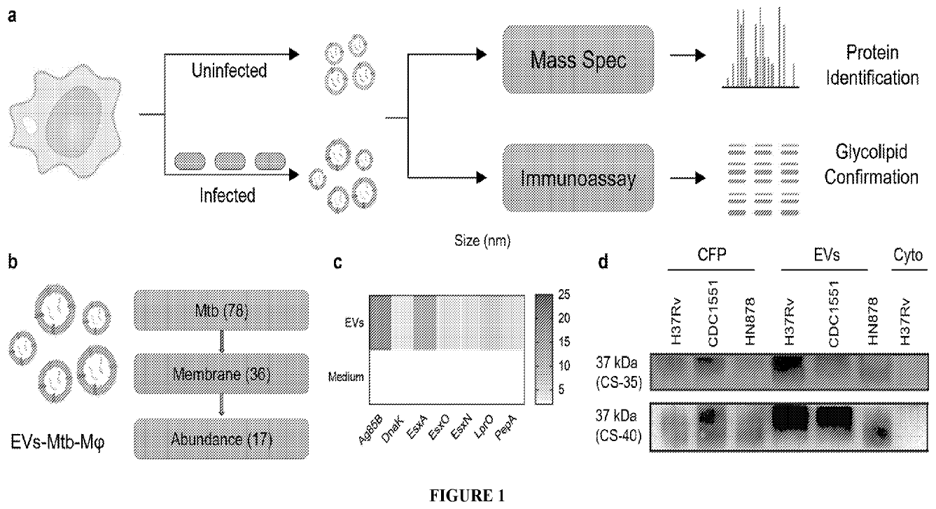

As shown in FIG. 1(b), it is found that 78 TB-derived proteins can be detected from TB-infected macrophages-derived EVs, among which 36 are membrane proteins that could be presented on the surface of the EV membrane for further detection without disrupting the EVs. 17 of the 36 proteins show high abundance and show promise as marker for further detection for TB diagnosis.

The TB-derived EV-specific markers identified herein include lipoarabinomannan (LAM), Antigen 85B (Ag85B), LAM carrier protein LprG and LpqH, Alpha-crystallin (HspX), DnaK, GroEL2, KatG, SodA and GlnA.

As shown in FIG. 1(c), several proteins, including Ag85B, DnaK, EsxA, EsxO, EsxN, LprO, and PepA, in TB-infected macrophages-derived EVs are significantly overexpressed compared to the culture medium of TB-infected macrophages. Among these proteins, Ag85b show the highest fold change in the EVs compared to that in the culture medium from TB-infected macrophages-derived EVs. Since LC-MS is not sensitive to LAM, a well-known glycolipid (37 KDa) that plays a critical role during TB infection, western blot was performed using two different anti-LAM antibodies (Clone ID: CS-35, CS-40) as the detection antibodies to confirm that LAM is also present in the EVs isolated from different strains of TB-infected macrophages. The results in FIG. 1(d) confirms the presence of LAM.

### 2. Verification of Markers

The markers discovered from screening experiment were then verified by western blot. (Data not shown)

### 3. Detecting Mtb Presence in EVs

We developed and optimized a nano plasmonic enhanced immunoassay to enable the highly sensitive detection of TB antigen on TB-infected macrophages-derived EVs. 1 μl of Mouse anti-CD81 antibody (5 ug/mL) was coated on patterned glass slides. After washing and blocking, 1 μL serum were added and incubated at 37° C. for 1 h followed by another 2 rounds of wash with PBS. 1 μL biotin functionalized Human anti-LAM antibody (1 μg/mL, A194-01) were then employed to capture the LAM on the EVs. The immobilized antibodies could then enable the conjugation of avidin-functionalized gold nanoparticles to generate scattering signal that can be observed with dark-field microscopy.

The Human anti-LAM antibodies (A194-01) used in this disclosure are monoclonal antibodies directed to epitopes found within lipoarabinomannan (LAM) and phosphatidyl-myo-inositol mannoside 6 (PIM6) for the diagnosis and treatment of MTB infections. Human monoclonal anti-LAM antibody A194-01 was isolated from cultured memory B cells obtained from a TB-infected patient, TB-194.

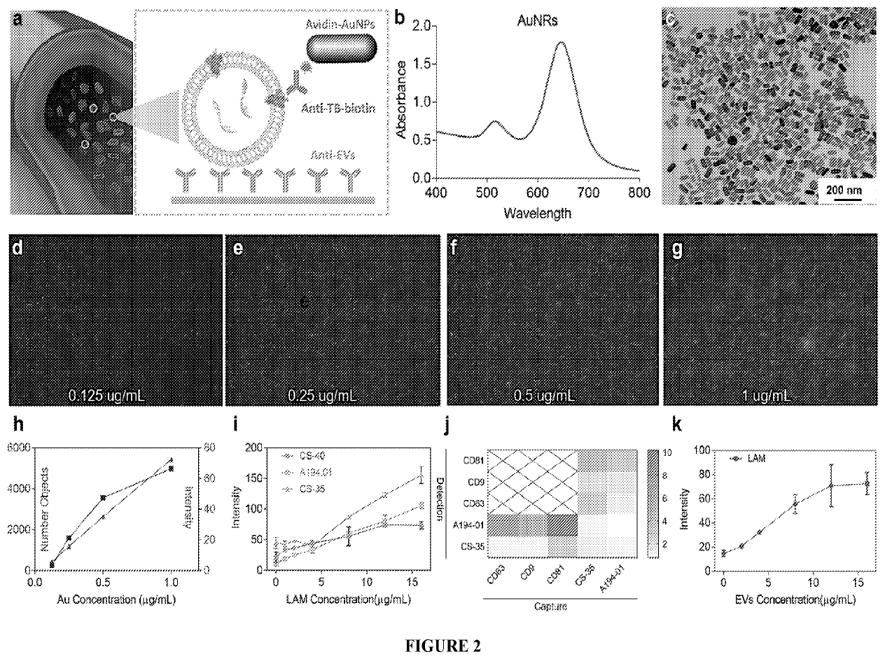

Please refer to FIG. 2a. EVs are captured on a glass slides with a capture antibody, and a detection antibody is used to bind the TB antigen on the EV membrane. Here the detection antibodies are further conjugated with gold nanorods (AuNRs) for the signal readout. The signal readouts of AuNRs can be seen in FIG. 2b.

AuNRs show an absorbance peak at 650 nm with uniform size distribution under TEM, as shown in FIG. 2c, and show gradually increased red scattering light with the increasing concentration of AuNRs under dark field microscopy, as shown in FIG. 2d-g. The signal is quantified by counting the number of the scattering object and the mean intensity of the pixels that form these images, and the mean intensity show a better linear response with the concentration of AuNRs, as shown in FIG. 2h.

As can be seen in FIG. 2(i), LAM was used as an exemplary target of this method, several anti-LAM antibodies (CS-35, CS-40 and A194-01) were tested. CS-35 shows the strongest signal with a relatively higher background, whereas A194-01 show the best linear response to LAM . Different combinations of capture antibodies and detection antibodies were further tested for the detection of 10 μg/mL TB-infected macrophages-derived EVs. Using anti-CD81 antibody as the capture antibody and anti-LAM antibody (A194-01) as the detection antibody resulted in the strong signal to blank (in the absence of EVs) ratio under the nano plasmonic enhanced immunoassay, as shown in FIG. 2j, and a dynamic range from 0-15 μg/mL is obtained for the detection of TB-infected macrophages-derived EVs under such antibody combination (FIG. 2k). This range provides practical detection limit for TB-specific EVs. This CD-81/A194-01 combination was further used to detect the EVs in serum samples for TB diagnosis.

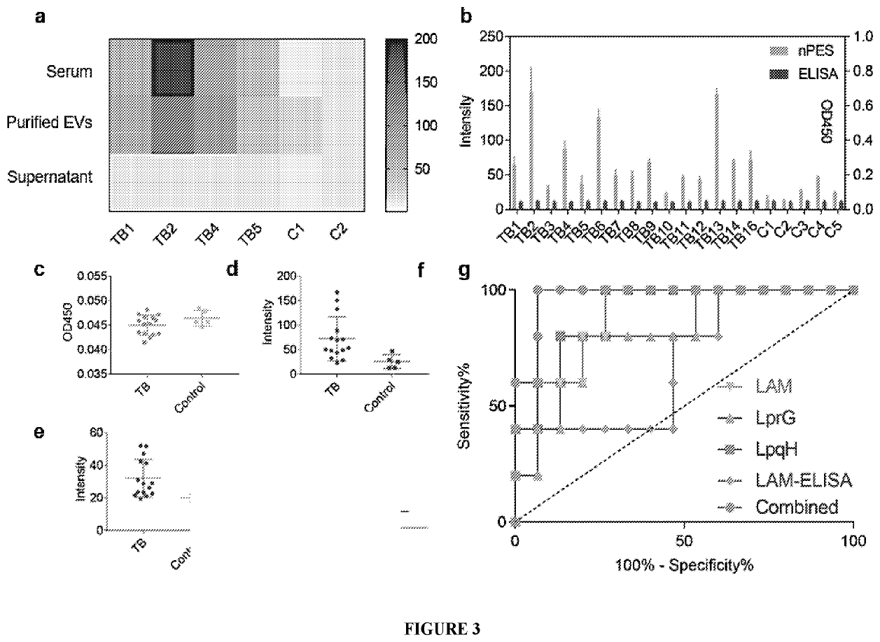

The method of this disclosure is also used to detect the TB antigens on EVs from the patient serum samples, and the results are shown in FIG. 3. In FIG. 3a, using anti-CD81 antibody as the capture antibody and the anti-LAM antibody (A194-01) as the detection antibody, both the whole serum and the purified EVs from these sample of TB patients show positive results.

15 untreated TB patient's serum and 5 control serum were further tested, and the result is shown in FIG. 3b. Most of these samples could generate a higher signal compared to the control. Traditional ELISA, on the contrary, fails to distinguish TB from Control.

Anti-LprG, anti-LpqH (two LAM carrier proteins) and anti-Ag85B were also used as the detection antibody in the nano plasmonic enhanced immunoassay, and the results are shown in FIGS. 3c-e. Significant differences are observed between TB and control when using the anti-LAM (FIG. 3c), anti-LprG (FIG. 3d) and anti-LpqH (FIG. 3e) as the detection antibody. The clear distinction between TB patients and control samples indicates that the method of this disclosure can effectively detect TB presence in samples.

The area under the curve (AUC) is a measure of the sensitivity/specificity of a testing method, and the greater the AUC indicates the better testing results. As can be seen in FIG. 3f, for traditional ELISA, the AUC is only 0.68, and for traditional Ag85B based nano plasmonic enhanced immunoassay the AUC is 0.627. In contrast, according to this disclosure, nano plasmonic enhanced immunoassay achieves AUC of 0.91, 0.90 and 0.83 using anti-LAM, anti-LprG and anti-LpqH as the detection antibody respectively, while using A194-01 as the capturing antibody. The results suggest that using the assay of this disclosure can achieve higher sensitivity and selectivity for effective TB diagnosis.

Additional samples were collected from 15 TB patients and tested. The results of the dark field images as well as the normalized intensity of test from 15 TB patients (TB1-14 and 16), 5 control serum samples (C1-5), and two exosome control samples (THP1 and THP1+CFP) were analyzed (not shown).

These results clearly indicate that the method and system of this disclosure can detect the presence of MTB in bodily fluid samples such as blood samples, and distinguish TB-negative from TB-positive samples. The method and system of this disclosure can also detect the presence of MTB in HIV-negative patients, which has not been demonstrated in the art. Also, the fast testing process ensures a quick and accurate diagnosis, reducing the wait time comparing to conventional culturing test

Additionally, image recognition software can also be optimized for the signal readout of immunoassay to lower the detection limit. It is also reported that different subtypes of EVs may increase the accuracy of detection, and therefore further focus is on the quantification of both LAM and other markers in different subtype of EVs form patient urine or serum samples. Lastly, correlation the concentration of LAM and host marker with the clinical information of TB patients can also provide insight on selecting proper treatment regimen.

### 4. Automated Detection of Mtb Presence in EVs

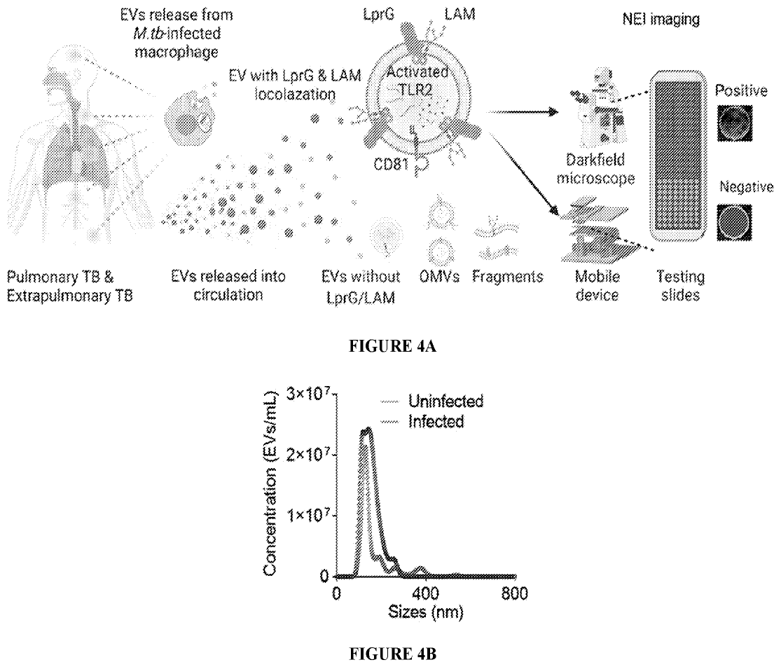

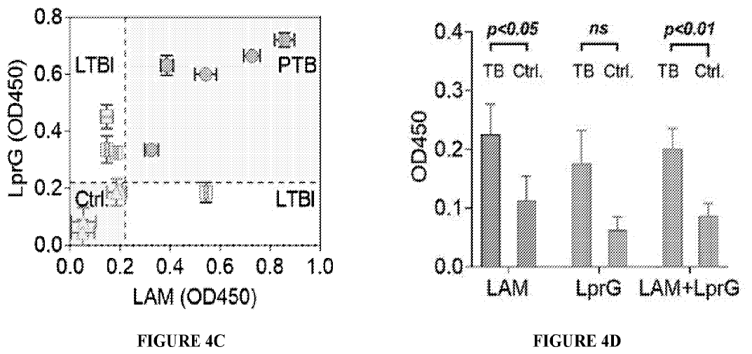

Automated nanoparticle-enhanced EV immunoassay (NEI) approach uses machine learning to detect EVs secreted by Mtb-infected cells (Mtb EVs) based on their surface expression of factors that are abundantly expressed on Mtb outer membrane. Lipoarabinomannan (LAM) is one of the target of this example because it accounts for 15% of Mtb biomass and regulates Mtb virulence. Another target is LprG, which is required for LAM distribution to the outer cell envelope. It is hypothesized that EVs secreted by Mtb-infected macrophages would exhibit surface expression of LAM and LprG that could serve as biomarkers of TB disease and that these Mtb EVs would be accumulated in the peripheral blood circulation, as well as other bodily fluid such as urine, saliva, etc., therefore allowing detection of both pulmonary and extrapulmonary TB. (FIG. 4A)

Through the NEI approach, it is discovered that when a combined Mtb EVs biomarker signature that integrated EV surface expression of LAM and LprG could differentiate active TB from latent TB infection (LTBI) in a non-human primate disease model, distinguishing both extrapulmonary and pulmonary TB from TB-negative pediatric controls, and diagnose TB in a diagnostically challenging population of hospitalized, severely immunosuppressed young children living with HIV who were at high risk of TB.

EVs secreted by Mtb-infected macrophages express LAM and LprG. To evaluate the potential ability of EV LAM and LprG expression to serve as biomarkers of Mtb infection or TB disease, we first examined the expression of these factors on EVs secreted by Mtb-infected macrophages. EVs isolated from macrophage cultures infected with or without the Mtb H37Rv reference strain revealed similar morphologies and size distributions (not shown), although Mtb-infected macrophages secreted markedly more EVs (FIG. 4B, 2.3×109 vs. 1.1×109 EVs/mL, p<0.01 by Kruskal-Wallis test). LAM and LprG demonstrated a significant EV enrichment upon analysis of equal amounts of cytosolic, cell membrane, and EV proteins extracts of macrophages infected with Mtb strains that exhibit variable growth rates and immunogenicity, indicating potential utility as strain-independent biomarkers of Mtb infection (not shown).

EV-ELISAs, a gold standard approach for the detection of EV surface makers25, also detected a dose-dependent increase in LAM and LprG signal (not shown) with serial dilutions of EVs produced by macrophages incubated with Mtb culture filtrate protein (CFP) extracts, verifying the surface expression of both markers.

The ability of serum EV LAM and LprG to discriminate between active TB, latent TB infection (LTBI), and healthy controls were next evaluated by analyzing serum EVs isolated from non-human primates who were Mtb naive or who developed LTBI or pulmonary TB (PTB) following Mtb exposure. NHP models were employed in this analysis to allow confident discrimination between active TB and LTBI, since it is difficult to distinguish LTBI from incipient or subclinical TB in human patient populations. Serum EVs isolated from Mtb naive NHPs had low non-specific background signal when analyzed by LAM and LprG EV-ELISAs (FIG. 4C), while serum EVs from the LTBI group demonstrated elevated expression of LAM or LprG, but not both, with most NHPs with LTBI revealing elevated LprG signal. However, serum EVs from the PTB group exhibited elevated expression of both LAM and LprG, which demonstrated a linear relationship, suggesting that these two factors could serve a composite biomarker for specific TB diagnosis. FIG. 4D is EV-ELISA for LAM, LprG and integrated LAM and LprG (LAM+LprG) expression on serum EVs of children with TB (N=10) and without evidence of TB. It is shown that LAM and LprG expressions are both significantly elevated in TB patients comparing to TB-negative controls. This further shows that the LAM and LprG are positive biomarkers for TB.

Similar analysis of EVs isolated from archived serum from a small, well-characterized case-control cohort of children (not shown) enrolled at the Vietnam National Lung Hospital (NLH) as part of a large, population-based TB surveillance study found that mean EV LAM and integrated EV LAM and LprG signal, but not EV LprG signal, differed between the TB and non-TB controls. Other Mtb-derived membrane-associated factors did not differ among serum EVs of these two groups (not shown), with the exception of LpqH, which had more signal overlap between the TB and non-TB groups than LAM or LprG.

### 5. NEI Detects LAM and LprG Expression on Mtb EVS With High Sensitivity

Given that target EVs could not be directly captured and analyzed from serum by EV ELISA, we investigated the feasibility of a darkfield microscope (DFM)-based NEI approach to improve target EV detection. Such assays can capture EVs from complex biological samples without time consuming isolation steps, and identify specific EV populations by detecting light scattered from gold nanorod (AuNR) probes bound to target biomarkers on their outer membranes. NEI signal is read by analysis of high- or low-magnification DFM images of sample wells. High-magnification analyses allow ultrasensitive detection, but require manual focusing to detect plasmonic signal from interacting nanoparticles which can introduce selection bias due to sampling of limited areas of the assay wells. Low-magnification DFM analyses can be automated, and are thus more suitable for clinical applications, but are subject to artifacts that can increase background and reduce sensitivity.

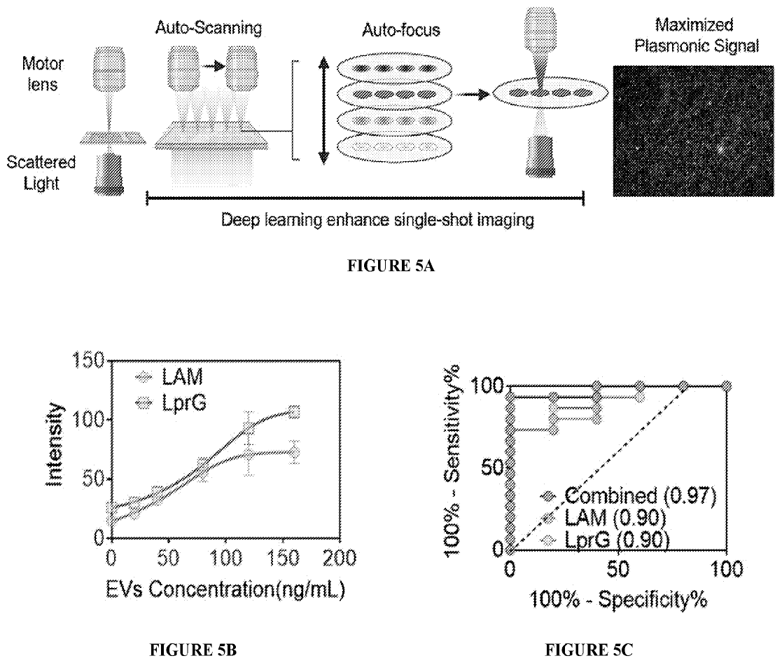

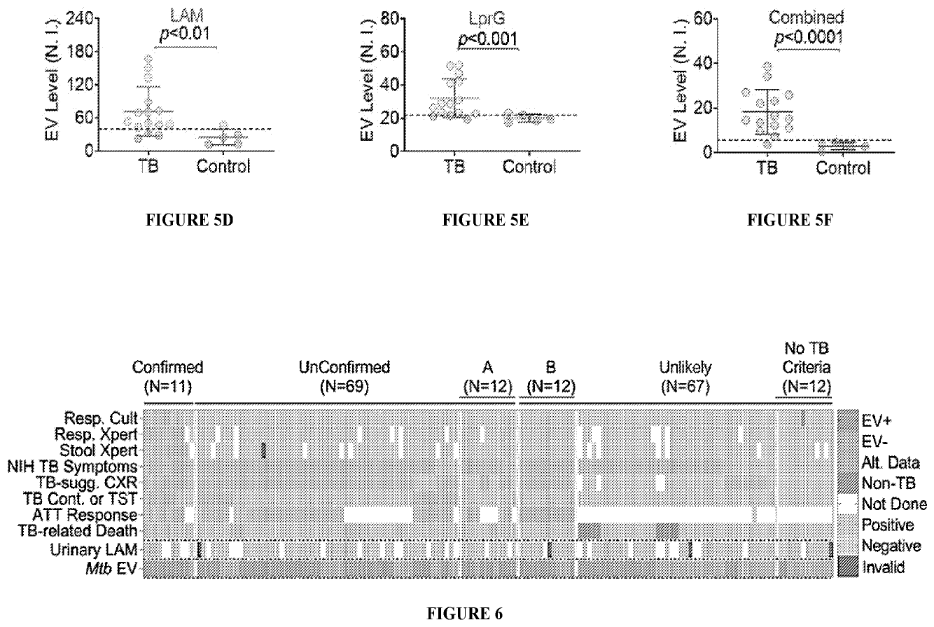

An NEI workflow (FIG. 5A) was then established, which allowed automatic capture of low-magnification DFM images that were processed with a custom noise reduction algorithm to reduce artifacts from serum aggregates, particulates, and surface scratches introduced during the assay. This algorithm detected and cropped the region of interest (ROI); converted each image into HSB (hue, saturation, brightness) color space; and normalized and applied thresholds to each HSB channel to identify AuNR-derived signal (not shown). This approach markedly reduced signal artifacts, as indicated by enhancement of AuNR signal in processed images and their pixel intensity maps (not shown). For signal detection, analysis of an AuNR dilution curve by quantifying positive pixels and mean pixel intensity found the latter approach had greater linearity with less variation (not shown).

To determine the probe concentration required to maximize the signal-to-noise ratio for target EVs at low concentration (near the limit of detection of the EV-ELISA), EVs isolated from Mtb-infected macrophages were captured by non-specific binding and hybridized with dilutions of anti-LAM antibody-conjugated AuNRs (not shown). Evaluation of capture and detection antibody pairs determined that anti-CD81 capture antibody and two anti-LAM (A194-01, Creative Biolabs) and anti-LprG (Clone B) detection antibodies produced the best signal-to-noise ratios (10.21 and 7.25, respectively), and good linearity and variability when employed over a broad range of EV concentrations (0-150 ng EV protein/mL; FIG. 5B).

NEI analysis of serum from the NHL discovery cohort found that serum EV LprG and LAM signal demonstrated similar ability to distinguish the TB and non-TB groups, but that EV LAM and LprG signal integrated via a logistic model (see Methods) had superior differential performance (FIG. 5C), as demonstrated when threshold from each of these analyses was used to differentiate TB and non-TB groups (FIG. 5D-F). This analysis revealed substantial signal overlap between the TB and non-TB groups near the thresholds for EV LAM or LprG signal, resulting in three false negatives and one false positive classification, while the integrated LAM and LprG signal produced a single false negative identification. NEI signal for LAM and LprG was detected in EV-enriched but not EV-depleted serum fractions, confirming that NEI signal was EV-specific (not shown). EV LAM and LprG signal did not distinguish TB- and non-TB cases when EV-ELISA was used to directly analyze serum from these groups, unlike results from EV enriched samples, demonstrating that EV-ELISA lacks the sensitivity required for a serum application. NEI signal did not differ when pediatric TB cases were stratified by age, sex or TB manifestation (pulmonary or extrapulmonary), suggesting that NEI signal served as a general marker of TB disease.

### 6. Mtb EV Biomarker Validation in a TB-Symptomatic HIV-Infected Pediatric Cohort

We next evaluated NEI assay performance in a diagnostically challenging cohort of children living with HIV at high risk for TB-related morbidity and mortality, often missed by respiratory sampling. This analysis employed serum collected from children with HIV in the multi-site Pediatric Urgent vs. post-Stabilization Highly active antiretroviral therapy (ART) initiation 145 trial (PUSH) in Kenya who were hospitalized and not yet initiated on ART at enrollment. Most children in this study exhibited severe immunosuppression as well as some symptoms consistent with TB. Children were retrospectively classified based on the 2015 NIH pediatric clinical TB case definitions as having confirmed TB if they had microbiologic evidence of TB (positive Mtb culture or positive Xpert results for respiratory or stool samples); unconfirmed TB (based on two or more of the following criteria: TB symptoms, abnormal CXR, close TB exposure or evidence of Mtb infection (i.e. positive TST), or positive response to TB treatment); or with unlikely TB if they lacked two criteria required for unconfirmed TB diagnosis. Children with confirmed and unconfirmed TB had high rates of symptoms (72.7% vs. 85.5%) and chest radiographic findings consistent with TB (90.9% vs. 80.6%), with most exhibiting positive findings for both TB criteria (63.6% vs. 81.1%; FIG. 6), although a subgroup of children was reclassified from unlikely to unconfirmed TB based on anti-TB treatment (ATT) response or TB-related death as determined by an expert review panel (FIG. 6, subgroup A). Children with unlikely TB frequently had symptoms or chest radiographic findings consistent with TB (65.7% vs. 31.8%), but a subgroup of children who had positive findings both criteria were classified as unlikely TB due to improvement of TB-related symptom following ART initiation without ATT initiation (FIG. 6, subgroup B).

NEI analyses performed using positive signal thresholds previously determined in the NLH discovery cohort by operators blinded to clinical information detected confirmed TB and unconfirmed TB with 90.9% and 72.5% diagnostic sensitivity (Table 2). NEI results exhibited similar sensitivity for unconfirmed TB cases with and without clinical TB diagnoses (FIG. 6), and detected all but one confirmed TB case (not shown). NEI results also detected a majority (52.7%) of children with unlikely TB who had at least one criteria required for unconfirmed TB diagnosis (Table 2). Since an age-matched low-risk cohort was not available, NEI diagnostic specificity was estimated in a subgroup of children with unlikely TB who did not have any have any clinical findings meeting the threshold of a feature of the NIH case definition. The Median Mtb EV signal in this group was significantly lower than in all other groups. Successful ATT response rates were high in children with confirmed and unconfirmed TB (87.2% vs. 66.7%; Table 1). Mortality was higher in children with confirmed vs. unconfirmed TB (33.3% vs. 8.5%; Table 2) after ATT initiation, but even higher (45.5%) in children with unconfirmed TB who were not diagnosed and treated during the study (31.9% 22/69), and lower (18.2%) in children with unlikely TB, although children with unlikely TB who died frequently had missing microbiologic or CXR results (FIG. 6, subgroup B). Urine LAM results exhibited poor diagnostic sensitivity for confirmed TB (42.8%; 3 of 7) and unconfirmed TB (5.6%; 3 of 53) cases with valid test results, and moderate specificity for unlikely TB cases (88.7%; 47 of 53), including children with no TB criteria (88.3%; 10 of 12).

### 7. Serum Mtb EV Signal Correspondence With TB Reclassifications and Treatment

Most children with unconfirmed TB were diagnosed using symptoms or chest radiographic data collected at the enrollment visit, but a subset of children were reclassified from unlikely TB to unconfirmed TB (17.9%; 12/67) based on subsequent findings (not shown), including deaths judged to be TB-related by an expert panel or positive responses to ATT. Reclassification decisions were made without knowledge of EV results, and most children reclassified from unlikely to unconfirmed TB (83.3%; 10/12) had detectable Mtb EV NEI signal at baseline and these values remained positive among children with sera available after their TB reclassification (not shown). Conversely, most unconfirmed TB cases reclassified as unlikely TB due to symptom improvement without ATT initiation had had positive Mtb EV NEI signal at baseline (75%; 9/12) that predominantly decreased at reclassification (88.9%; 8/9; data not shown). Taken together, these findings suggest that longitudinal decreases in serum NEI signal that reflected Mtb EV decreases were associated with resolution of TB disease with ATT response and perhaps clearance or containment with immune reconstitution.

### 8. Design and Validation of a Portable DFM Device for Mtb EV Assay Signal Readout

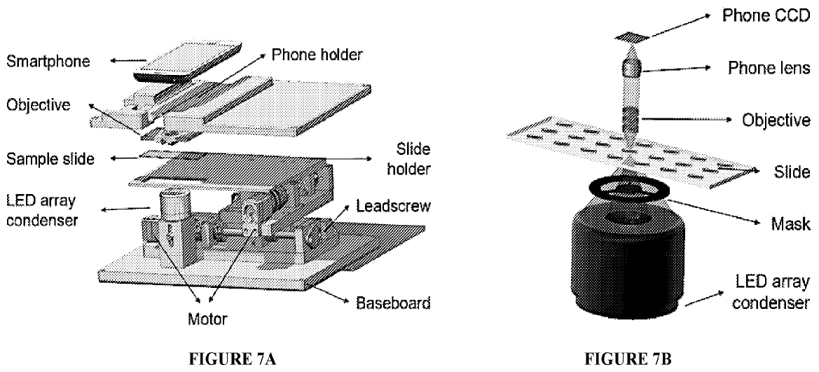

To adapt this assay platform for resource-limited settings where TB is prevalent, we employed a 3D printer to fabricate an inexpensive and portable smartphone-based DFM device that can scan a 144 well assay slide in 5 minutes (FIG. 7A). This device employed an aluminum slide holder to add stability and maintain focus during automated scanning and an illumination mask to block stray light and improve DFM signal intensity (FIG. 7B). A smartphone app developed for this device allowed manual centering and focusing of the first slide well, after which the app automatically centered, focused, and captured images of the remaining wells, saving all images to separate files for download and analysis. This device produced results similar to benchtop DFM results when employed to read NEI data from the discovery cohort, accurately identifying 73% ( 11/15; versus 80%; 12/15) of TB cases and 87% ( 13/15; versus 93%; 14/15) of non-TB cases. Normalized NEI signal intensity for integrated and single biomarker signal was also similar on these two devices (data not shown).

The novel NEI approach can be used to detect Mtb EV biomarkers in bodily fluid to permit rapid diagnosis of TB by refining a standard NEI approach to permit automated image capture and ultrasensitive detection of the target signal. The method and system of this disclosure can be further applicable to other bacterial pathogens that can be found in EVs. This approach employed a workflow suitable for use in clinical settings to detect TB in pediatric populations at high risk of TB, where TB is often missed by tests employing respiratory specimens. This assay directly captured EVs from serum or other bodily fluids, eliminating the common EV immunoassay requirement for purified EV samples, which are normally isolated by methods that involve tradeoffs between time, labor, expense and EV yield, purity and integrity that limit their clinical feasibility. Since there are currently no widely accepted EV biomarkers of TB, this study evaluated the potential diagnostic utility of measuring serum levels of EVs expressing two Mtb-derived factors associated with TB virulence, LAM and LprG, finding that EV expression of these markers was enhanced in TB cases, and that multiplex detection of these two factors could differentiate LTBI and TB in an NHP model. LAM and LprG are both highly expressed in Mtb bacilli, which could contribute to their diagnostic performance. However, other abundant Mtb-derived factors identified as potential targets by a literature search did not exhibit similar capacity to distinguish TB and non-TB cases, including the membrane protein Ag85b, a mycolyl transferase required for efficient biosynthesis of the Mtb cell wall. In this analysis, only the Mtb membrane protein LpqH exhibited a significant difference between these two groups, although it is not clear if the secreted Mtb proteins analyzed in this study (CFP10, ESAT6, MPT51, and MPT64) form stable membrane interactions that would permit their detection on serum EVs.

NEI analysis of an NHP model of LTBI and TB indicated that serum EV expression of both LAM and LprG was required to distinguish TB from LTBI. LAM is a virulence factor that is expressed on the Mtb outer cell wall where it can bind to the macrophage mannose receptor to facilitate cellular entry of Mtb bacilli in host phagocytes, and inhibit phagosome-lysosome fusion and modulate the immune response to promote continued intracellular survival of Mtb required for TB development. LprG plays an essential role in the localization of LAM to the outer cell envelope of Mtb bacilli, and LprG null Mtb mutants exhibit reduced LAM surface expression and virulence, decreased Mtb entry into host macrophages, reduced biogenesis and/or integrity of the Mtb cell envelope, failure to inhibit phagosome-lysosome fusion, and reduced intracellular replication rates. LprG is thus essential for LAM activity, since LprG deficiency attenuates Mtb virulence without altering LAM expression. LprG expression might thus be expected to be downregulated in NHPs with LTBI cases; however, our results suggest the opposite case: downregulation LAM and upregulation of LprG expression on serum EVs from NHPs diagnosed with LTBI, whereas both markers were elevated on serum EVs from NHPs diagnosed with TB. Several mechanisms could explain this finding, including downregulation of LAM expression or inhibition of LprG of LprG activity to limit LAM transport, both of which would be expected to limit LAM expression on the Mtb outer membrane and, presumably, LAM expression of EVs secreted by Mtb-infected cells during LTBI. However, the mechanism(s) responsible for this differential expression and its functional significance is unclear and merits further study, including replication in human studies with well-defined LTBI and TB cohorts.

NEI analysis of Mil) EVs exhibited good diagnostic sensitivity for both confirmed and unconfirmed TB in a diagnostically challenging population of children living with HIV including children not clinically diagnosed during the parent study despite extensive TB work-up. NEI diagnostic sensitivity in this group is of particular relevance since mortality was more than five times higher in untreated children who were not diagnosed with TB during clinical evaluation than those who were diagnosed and treated at evaluation, in keeping with reported high mortality rates in children who do not receive ATT due to missed diagnoses. Notably, serum from several children exhibited positive Mtb EV signal prior to their TB diagnosis by clinical findings, suggesting that serum Mtb EV signal have potential as a means for early TB diagnosis, which is of particular importance in this population since a third of the children identified in this manner died at or shortly after their diagnosis by conventional means.

NEI Mtb EV signal markedly decreased following ATT initiation for both confirmed and unconfirmed TB cases, in agreement with TB symptom improvement, suggesting that Mtb EV level might be useful as a surrogate for ATT response. Similar decreases were also observed in a subset of children who met the criteria for unconfirmed TB when evaluated by their baseline data, but who were reclassified as unlikely TB due to improvement of their TB suggestive symptoms following ART initiation without ATT initiation. NEI Mtb EV signal decreases observed in all these cases suggest that children may have had nascent TB which was at least partially contained by improved immune function following ART initiation.

As discussed above, by first extracting EVs from a bodily fluid sample, it is possible to significantly increase the sensitivity and specificity of the test by using NEI with antibodies against LAM and LprG. The method can accurately detect the presence of Mtb in the bodily fluid sample, and based on the detected expression of LAM and LprG, one can also determine whether the infection is latent or active. The combination of NEI and dark field microscopy allows the highly sensitive Mtb detection without the need of further enriching the EV concentration in the sample. The automated NEI detection further increases the efficiency of detection with high accuracy.

The following methods and materials are used in this disclosure.

### Methods

Mtb Culture. Mtb strains H37Rv, CDC1551 and HN878 were obtained from Houston Methodist Hospital and cultured at 37° C. in roller bottles to mid-log phase (OD600=0.5-0.6) in a pH 7.0 minimal protein-free medium containing 0.1% (v/v) glycerol, 1 g/L KH₂PO₄, 2.5 g/L Na₂HPO₄, 0.5 g/L asparagine, 50 mg/L ferric ammonium citrate, 0.5 g/L MgSO₄×7 H₂O, 0.5 mg/L CaCl₂, and 0.1 mg/ L ZnSO₄, with or without 0.05% tyloxapol (v/v).

THP-1 cell culture and differentiation. THP-1 monocytes were purchased from the American Type Culture Collection (ATCC; Manassas, Va.) and cultured at 37° C. in a 5% CO2 incubator in RPMI 1640 supplemented with 10% FBS. Macrophage differentiation was performed by incubating 10 T175 flasks containing ˜2.5×10⁷ viable THP-1 monocytes/flask (5-7×10⁵ cells/mL; ≥95% viability) for 24 with nM PMA (Sigma Aldrich, 389 USA), after which the flasks were washed 3× with 37° C. PBS to remove PMA and non-adherent cells before culturing the adherent, differentiated THP-1 macrophages in RPMI 1640 supplemented with 10% FBS.

For experiments using Mtb-infected macrophages, mid-log phase Mtb H37Rv, CDC1551 and HN878 cultures (10 mL) were pelleted by centrifugation at 3000 g for 10 min at 4° C., and resulting bacterial pellets were suspended in 10 mL of RPMI 1640/10% FBS without penicillin and streptomycin, de-clumped using a brief sonication step, and passed 10 times through syringe fitted with 27-gauge needle (VWR, Norm-Ject, USA). Mtb suspensions were then mixed with an additional 10 mL of antibiotic-free RPMI 1640/10% FBS, and 0.1 mL aliquots of suspensions were added to T175 flasks containing ˜2.5'10⁷ differentiated THP-1 macrophages cultured in 20 mL antibiotic-free RPMI 1640/10% FBS to obtain a multiplicity of infection (MOI) of 10. After 4 h incubation, cell cultures were washed 3× with 37° C. PBS to remove extracellular Mtb bacilli and cultured in RPMI 1640 without FBS for 48 h, after which culture supernatant was passed through 0.22 um filters to remove Mtb bacilli and generate samples for EV and soluble protein analyses. Culture filtrates were stored at −80° C. while aliquots were inoculated into mycobacterial growth indicator tubes and assessed for Mtb growth after 3-4 weeks of culture to confirm the absence of viable Mtb bacilli remained in these samples. Cultured macrophages were recovered by trypsin digestion and split into samples that were analyzed for viability and employed to generate cell lysates for Western blot analysis of target proteins.

For experiments using culture filtrate protein (CFP), aliquots containing 100 μg CFP were added to T175 flasks containing ˜2.5×10⁷ differentiated THP-1 macrophages cultured in 20 mL RPMI 1640/10% FBS. After 4 h incubation, cell cultures were washed 3× with 37° C. PBS to remove extracellular CFP and cultured in RPMI 1640 without FBS for 48 h, after which culture supernatant were collected to generate samples for EV and soluble protein analyses. Cultured macrophages were recovered by trypsin digestion and split into samples that were analyzed for viability and employed to generate cell lysates for Western blot analysis of target proteins.

EV isolation and characterization. Cryopreserved serum aliquots (1 mL) that were processed for EV isolation were rapidly thawed in a 37° C. water bath, vortexed for 3 s, then 300 μL aliquots were transferred to 2 mL centrifugation tubes, mixed with 1.2 mL PBS, and then centrifuged at 1200 rpm for 15 min at 4° C. to pellet large particulates and debris. Supernatants were centrifuged at 2,000 g for 30 min at 4° C., passed through a 0.45 μm filter, and centrifuged twice at 10,000 rpm and 4° C. for 45 min, and then at 110,000 g and 4° C. for 90 min. Resulting EV pellets were suspended in 1 mL PBS, centrifuged at 110,000 g and 4° C. for 3 h, and then suspended in 50 μL PBS and characterized by bicinchoninic acid (BCA) assay and NanoSight nanoparticle tracking analysis (Malvern Panalytical, USA; 5 μg/mL with 5 replicates) to determine protein content and EV concentration and size distribution. Cell culture supernatants used for EV isolation were concentrated by 15 min centrifugation at 4° C. and 3000 rpm on 50 mL 10 kDa Copolymer Styrene ultrafiltration tubes (Millipore Sigma, USA) to concentrate 200 mL of starting material to 100 μL, after which these concentrated samples were processed for EVs isolation, and characterized, as described above. For transmission electron microscope analyses, carbon-coated Cu grids (400 mesh), incubated for 30 s at room temperature with 5 μL EV aliquots containing 1 mg/mL protein, blotted with filter paper to remove excess liquid, incubated with 2 μL of 1% osmium tetroxide for 2 min at room temperature to stain membranes, blotted to remove this solution, and imaged at the indicated magnification using a Tecani F30 microscope (FEI Company, USA) with an accelerating voltage of 200 kV.

EV ELISA analyses. Standard EV capture plates used for EV-ELISA were generated by adding 100 μL aliquots of mouse antibody specific for human CD81 (BioLegend, US) to each well of 96 well microtiter plates (5 μg antibody/mL in PBS) and incubating these plates for 16 h at room temperature. Wells were then washed 3× with 260 μL PBST (30 s per wash), then blocked by incubation with 250 μL blocking buffer (1% wgt/vol BSA in PBST) for 2 h at 37° C., and again washed 3× with PBST. To analyze optimal EV capture conditions for NEI, wells were alternately incubated with 100 μL aliquots of mouse antibodies specific for EV surface markers including CD9, CD63, CD81 and different clones of anti-LAM/LprG antibodies.

For EV-ELISAs, each well was incubated overnight at 4° C. with 100 μL of isolated EVs or serum, washed 3× with PBST, and then incubated with 100 μL PBST containing 1 μg/mL of the indicated detection antibodies for 1 h at 37° C., washed 3× with PBST, then incubated with 100 μL PBST containing 0.5 μg/mL HRP-labeled goat anti-mouse/human IgG (Jackson Immune lab, USA) for 30 mins at 37° C., and washed 3× with PBST. After the final wash step, wells were incubated with 50 μL TMB (Sigma, US) for 10 mins at room temperature and then mixed with 50 μL of 2 M H₂SO₄ to stop the reaction, after which target EV for each well were read using a microplate reader (Tecan, Ultra, Switzerland) to measure OD 450 values after subtracting OD 650 signal.

Non-human primate (NHP) Mtb infection and sample collection. Cryopreserved NHP plasma analyzed in this study was obtained archived material from NHPs infected with Mtb in previously reported studies35. Briefly, specific-pathogen free, retrovirus-free, mycobacteria-naive, adult rhesus macaques were assigned to three experimental groups that received different Mtb exposures (none, low and high). Samples for the negative control (Mtb naive) cohort were obtained from four uninfected rhesus macaques who were not exposed to Mtb during the study period. Samples for the latent TB infection (LTBI) cohort were obtained from four rhesus macaques subjected to low-dose Mtb aerosol exposure event (˜10 CFU of Mtb CDC1551), which had positive results for tuberculin skin tests (TST) within a month after exposure but did not exhibit any signs of TB, instead maintaining asymptomatic LTBI-like infections throughout the study (˜22 weeks). Samples for the TB cohort were obtained from five rhesus macaques that were subjected to a high-dose Mtb aerosol event (˜200 CFU of Mtb CDC1551), which developed active TB disease characterized by weight loss, pyrexia, elevated serum C-reactive protein levels, elevated chest radiograph scores consistent with TB, detectable Mtb CFU levels in bronchoalveolar lavage fluid, higher lung bacterial burden, and associated lung pathology at the study endpoint. Lung tissue collected at the study endpoint was randomly sampled by pathologists blinded to animal treatment using a grid, as described previously.

NEI analyses. EV capture slides used for NEI analyses were generated by adding 1 μL aliquots of mouse antibody specific for human CD81 EV capture or indicated antibodies (5 μg/mL) to each position of a 144 well mask affixed to a microscope slide and incubating these slides for 16 h at 4° C. All incubation steps in this analysis were performed in a humidified chamber to reduce evaporation effects. Following capture antibody binding, slides were washed 3× with PBST, and blocked with 1 μL/well SuperBlock™ (PBS) Blocking Buffer (Cytiva, USA) for 1 h at 4° C., and then washed 3× with PB ST before incubation with 1 μL of serum or isolated EV samples. Serum samples were centrifugated at 10000 g for 20 minutes to remove large debris. Serum or EV samples were incubated on EV capture slides for 16 h at 4° C. then washed 3× with PBS, hybridized for 1 h at 37° C. with 1 μl of the specified biotinylated detection antibody (1 μg/mL), washed 3× with PBS, and incubated for 1 h at 37° C. with neutravidin-functionalized AuNR (Nanopartz, USA) at the indicated concentrations. After the AuNR incubation step, slides were washed once with PB ST and distilled water to remove unbound particles before they were subjected to DFM photography and image analysis.

DFM noise reduction algorithm. NEI signal was analyzed using a custom designed algorithm that identified the first identified area of each assay to be analyzed and then and subtracted DFM artifacts and background signal. This algorithm detected the high-intensity boundary of each well, calculated the center position of the circular region formed by their high-intensity boundaries, and then selected the central area of this region to avoid potential “coffee-ring effects” caused by the accumulation of residual AuNRs at the edges of these regions after the final wash step. To remove DFM artifacts and background, images were converted to HSB (Hue, Saturation, Brightness) color space, and the values in each channel from the 16-bit images were normalized to a 0 to 1 range since hue is measured in degrees (0° to 360°). The hue and saturation channels were employed to identify AuNR signal and remove artefacts using a sets of training images from slides coated with pure AuNRs and mixtures of AuNRs and human serum. Hue was employed to identify pixels that matched the AuNR scattering range and saturation was employed to evaluated the color intensity (purity) of pixels that matched the AuNR range. Pixels with Hue channel values outside the red scattering range of AuNR signal (≥0.8 and ≤0.05 on the hue color wheel) were excluded as were pixels with very low saturation values (≤0.05). 476 AuNR signal in processed images was measured using MATLAB (software version 2020a), using the parameters.

### Clinical Cohort Evaluations

NLH cohort. Specimens and associated clinical data were collected from 20 children at the Vietnam National Lung Hospital (NLH), who consecutively came to the Department of Pediatrics at the NLH in Ha Noi, Vietnam for clinical assessment and medical evaluation. The NLH cohort included children <17 years of age who were seen by the clinicians at the NLH, Department of Pediatrics and provided written informed consent from the parent or legal guardian. Children were excluded from participation if they were ≤17 years of age, had a laboratory-documented anemia (Hemoglobin<9 mg/dL), or where informed consent was not obtained for all study procedures. Children were not excluded if they currently had HIV-infection or had receive antiretroviral therapy (ART). Controls were children with broncho-pulmonary diagnoses other than TB who had negative QuantiFERON-TB Gold Plus (QFT; Hilden, Germany), Xpert GeneXpert MTB/RIF (Xpert; Cepheid, USA), and Mtb culture results, and had TB ruled out clinical assessment by experienced pediatric TB specialists. TB cases were defined by positive Mtb culture and/or GeneXpert MTB/RIF (Xpert; Cepheid, USA) results for pulmonary or extra-pulmonary specimens. The protocol was approved by the NLH Institutional Review Board.

PUSH cohort. The Pediatric Urgent vs. post-Stabilization highly active antiretroviral therapy (HAART) initiation trial (PUSH) study was a randomized controlled trial (NCT02063880) performed in Kenya that evaluated whether urgent (<48 hours) vs. post-stabilization antiretroviral therapy (7-14 days) improved survival in hospitalized HIV-infected children aged <12 years. At enrollment, children were systematically screened for TB symptoms and TB exposure and underwent intensive TB evaluation including chest radiograph, sputum or gastric aspirates (GA) for Xpert and culture, urine for lipoarabinomannan (LAM) antigen testing, and stool for Xpert, irrespective of TB symptoms. Serum was collected and cryopreserved at enrollment and at 2, 4, 12, and 24 weeks after enrollment. CXRs were read by a radiologist using standardized reporting forms to identify findings suggestive of TB developed by the South African Tuberculosis Vaccine Initiative. A positive tuberculin skin test (TST) result was defined as induration >5 mm. Diagnostic results were available to study clinicians and TB treatment was initiated at their discretion, per Kenyan guidelines.

Children were post-hoc categorized as confirmed TB (Mtb positive culture or Xpert result for a respiratory or stool sample), unconfirmed TB (>2 or more of the following: TB symptoms, abnormal CXR consistent with TB, TST positive or known TB exposure, and/or TB treatment response), or unlikely TB (not meeting other criteria) based on the NIH international consensus clinical case definitions for pediatric TB (not shown). TB symptoms were defined as a cough lasting >2 weeks, weight loss/failure to thrive, fever lasting >1 week, and/or lethargy lasting >1 week. For children who died during the study, an expert panel reviewed cases and came to a consensus regarding whether death was considered to be likely, possibly, or unlikely related to TB. Death considered likely or possibly related to TB was employed as an additional criterion for unconfirmed TB classification (Table S6). For the purpose of assay evaluation, children with unconfirmed TB were further stratified by whether they received a clinical TB diagnosis and ATT or did not have a clinical TB diagnosis and did not start ATT. Children categorized as unlikely TB were further stratified by the presence or absence of features employed for unconfirmed TB diagnosis.

Children with cryopreserved serum available within 2 weeks of TB diagnosis, including within 2 weeks ATT initiation, were evaluated to estimate TB diagnostic performance of Mtb EV NEI results. Children who initiated TB treatment with samples within 2 week of TB treatment initiation and at least one additional subsequent sample were eligible for the treatment response analyses.

Participant characteristics were summarized by frequency and proportion for categorical variables, and by median and interquartile range (IQR) for continuous variables (Table 1). We estimated NEI EV performance to detect TB by sensitivity and specificity using 95% confidence intervals (CI) assuming a binomial distribution stratified by NIH pediatric TB clinical case definitions (Table 2). Notably many children in the unlikely TB category had either CXR suggestive of TB or TB symptoms (but not meeting unconfirmed TB criteria) therefore we further stratified the unlikely TB category by the presence or absence of abnormal CXR and TB symptoms to identify a potentially more appropriate negative control group. Median Mtb NEI EV levels were evaluated by Wilcoxon rank sum test compared to the reference of Unlikely TB with no TB symptoms and negative CXR. For treatment response, median Mtb NEI EV levels at TB treatment initiation and at latest available sample were evaluated by paired Wilcoxon signed-rank test (median time between TB treatment initiation and latest available sample 5.5 months (IQR 3.1-5.7).

Study CXR were not performed in 24 children (4 confirmed, 2 unconfirmed, and 18 unlikely). Additional CXR results were extracted from hospital medical records to inform TB classifications. This approach identified hospital CXR data for 14 of the 24 children with missing study CXR data. If hospital CXR results noted characteristics compatible with TB (SATVI criteria), we included this information to determine TB classification (3 changed from unlikely TB to unconfirmed TB using this information). An additional XX children with missing CXR and clinical pneumonia were assumed to have implied CXR findings consistent with TB (i.e. would have likely have had TB suggestive CXR findings if CXR results had been available. If hospital CXR was only noted as “abnormal” without specific TB CXR features defined, we did not consider this sufficient for TB classification.

Portable DFM device. The mobile DFM slide scanning system (270×190×106 mm) has a slide scanning range of 82 mm×38 mm, and consists of a mechanical scanning system with two step motors, a dark field light source, an interchangeable smart phone with a miniaturized objective, an IOIO-OTG board (DEV-13613,

SparkFun Electronics, Colorado) and two motor driver boards (ROB-12779, SparkFun Electronics, Colorado) to control the step motors and communicate with the smart phone component via Bluetooth. This system employs two lithium batteries: a 7.6V 3500 mAh Lipo Battery (3.35×1.97×0.55 inches, GAONENG, China) supplies power for the electronics, and a 11.1V 3200 mAh Lipo Battery (5.16×1.73×0.67 inches, HOOVO, China) supplies power for the motors. Most of the additional parts were printed with a commercial 3D printer (Objet 30 prime, Stratasys, Israel), but the slide holder employed to ensure the stability of the slide during the scanning process was fabricated from an aluminum plate using a computer numerical control milling machine. The dark field light source consists of an integrated dark field condenser containing an illumination numerical aperture (NA) ranging from 0.7 to 0.9 and an array of 3 mm white LEDs with a viewing angle of 30 degrees, and employs a mask placed in front of the condenser to block stray light and improve the contrast of dark field images. The objective consists of three identical doublets; it has a focal length of 3.4 mm, NA of 0.25, working distance of 1 mm, and field of view of 1.6×1.6 mm. The camera of the mobile phone component of this device, a Moto G6, had a 3.95 mm focal length, a working F-number of 1.8, and a sensor with 12 megapixel resolution (3072×4096 pixels, pixel size: 1.4 μm).

Statistical Analysis. GraphPad Prism (Version 7.0) was employed to generate figures, heatmaps and perform statistical analyses. Potential differences between groups were analyzed by parametric or non-parametric (Kruskal-Wallis) one-way analysis of variance (ANOVA) with multiple comparison tests, Wilcoxon signed-rank test, student t-tests or Mann-Whitney U-tests, as appropriate. Logistic regression was performed to evaluate combinations of LAM and LprG signal that could best differentiate positive and negative control groups to establish a diagnostic equation, with hypothesis testing performed with a two-sided α of 0.05 for the significance level. Both characteristics were entered into this logistic regression model without other covariates. All analyses were done with SAS® OnDemand version 9.4 for Academics (https://welcome.oda.sas.com/login). Data are presented as mean±SD unless noted otherwise.

The following references are incorporated by reference in their entirety for all purposes.

Zumla, Alimuddin, et al. “WHO's 2013 global report on tuberculosis: successes, threats, and opportunities.” The Lancet 382.9907 (2013): 1765-1767.

Zumla, Alimuddin, et al. “The WHO 2014 global tuberculosis report—further to go.” The Lancet Global Health 3.1 (2015): e10-e12.

Bass Jr, J. B., et al. “Diagnostic standards and classification of tuberculosis.” American Journal of Respiratory and Critical Care Medicine 142.3 (1990): 725-735.

Boehme, Catharina C., et al. “Rapid molecular detection of tuberculosis and rifampin resistance.” New England Journal of Medicine 363.11 (2010): 1005-1015.

Yerlikaya, Seda, et al. “A tuberculosis biomarker database: the key to novel TB diagnostics.” International Journal of Infectious Diseases 56 (2017): 253-257.

Paris, Luisa, et al. “Urine lipoarabinomannan glycan in HIV-negative patients with pulmonary tuberculosis correlates with disease severity.” Science translational medicine 9.420 (2017): eaa12807.

Raviglione, Mario, and GiorgiaSulis. “Tuberculosis 2015: burden, challenges and strategy for control and elimination.” Infectious disease reports 8.2 (2016).

C. M. Denkinger, S. V. Kik, D. M. Cirillo, M. Casenghi, T. Shinnick, K. Weyer, C. Gilpin, C. C. Boehme, M. Schito, M. Kimerling, M. Pai, Defining the needs for next generation assays for tuberculosis. J. Infect. Dis. 211, S29—S38 (2015).

Raposo, Graça, and Willem Stoorvogel. “Extracellular vesicles: exosomes, microvesicles, and friends.” J Cell Biol 200.4 (2013): 373-383.

Liang, Kai, et al. “Nanoplasmonic quantification of tumour-derived extracellular vesicles in plasma microsamples for diagnosis and treatment monitoring.” Nature biomedical engineering 1.4 (2017): 0021.

Sun, Dali, and Tony Y. Hu. “A low cost mobile phone dark-field microscope for nanoparticle-based quantitative studies.” Biosensors and Bioelectronics 99 (2018): 513-518.

Russell, David G. “Who puts the tubercle in tuberculosis?.” Nature Reviews Microbiology 5.1 (2007): 39.

Athman, Jaffre J., et al. “Bacterial membrane vesicles mediate the release of Mycobacterium tuberculosis lipoglycans and lipoproteins from infected macrophages.” The Journal of Immunology 195.3 (2015): 1044-1053.

TABLE 1
Baseline characteristics of hospitalized ART-naïve children living with HIV who were at high risk for TB

| | | Total | | Confirmed TB | | Unconfirmed TB | | Unlikely TB | |
|---|---|---|---|---|---|---|---|---|---|
| | | N = 147 | | N = 11 | | N = 69 | | N = 67 | |
| | | median (IQR) | | median (IQR) | | median (IQR) | | median (IQR) | |
| Characteristics | N | or n (%) | | or n (%) | | or n (%) | | or n (%) | |
| Demographic | | | | | | | | | |
| Age (years) | 147 | 1.8 | (0.8-4.4) | 4.0 | (1.2-7.2) | 1.8 | (0.8-4.3) | 1.6 | (0.7-3.9) |
| Female sex | 147 | 70 | (47.6) | 4 | (36.4) | 33 | (47.8) | 33 | (49.3) |
| Clinical presentation | | | | | | | | | |
| CD4 cell count (cells/μl) | 146 | 727.5 | (317-1240) | 438 | (104-799) | 714 | (216.5-1361) | 757 | (495-1445) |
| CD4 % | 146 | 15 | (9-22) | 11 | (6-25) | 13 | (6.5-18.8) | 18 | (11.4-24.5) |
| Severe immunosuppression ^{a} | 146 | 102 | (69.9) | 8 | (72.7) | 53 | (77.9) | 41 | (61.2) |
| Wasted (WHZ < −2) ^{b} | 114 | 45 | (39.5) | 5 | (71.4) | 24 | (44.4) | 16 | (30.2) |
| Underweight (WAZ < −2) | 147 | 95 | (64.6) | 7 | (63.6) | 50 | (72.5) | 38 | (56.7) |
| TB features | | | | | | | | | |
| NIH criteria signs/symptoms of TB ^{c} | 147 | 111 | (75.5) | 8 | (72.7) | 59 | (85.5) | 44 | (65.7) |
| TST ≥ 5 mm | 134 | 7 | (5.2) | 1 | (10.0) | 5 | (8.2) | 1 | (1.6) |
| TB contact | 147 | 25 | (17.0) | 6 | (54.6) | 16 | (23.2) | 3 | (4.5) |
| CXR suggestive of TB | 141 | 84 | (59.6) | 10 | (90.9) | 54 | (80.6) | 20 | (31.8) |
| Positive respiratory culture or Xpert ^{d} | 147 | 10 | (6.8) | 10 | (90.9) | 0 | | 0 | |
| Positive stool Xpert ^{e} | 130 | 7 | (5.4) | 7 | (70.0) | 0 | | 0 | |
| Positive urine LAM ^{f} | 116 | 12 | (10.3) | 3 | (42.9) | 3 | (5.6) | 6 | (10.9) |
| ATT initiated | 147 | 57 | (33.8) | 9 | (81.8) ^{g} | 47 | (68.1) | 1 | (1.5) |
| Positive ATT response | 57 | 47 | (82.5) | 6 | (66.7) | 41 | (87.2) | 0 | |
| Deaths ^{h} | 147 | 29 | (19.7) | 5 | (45.5) | 14 | (20.3) | 10 | (14.9) |

N: number of participants with results; n: number of 588 participants with positive results.
^{a} According to WHO age-specified CD4% cut-offs for severe immunosuppression, or CD4 count in absence of CD4 % data (<12 months: <25%/<1500 cells/μl; 12-35 months: <20%/<750 cells/μl; >36 months: <15%/<350 cells/μl).
^{b} Among children 5 years and under: WHZ < −2 or MUAC < 12.5 cm
^{c} Persistent cough (≥14 days), fever (≥7 days), failure to thrive, or lethargy (≥7 days). Failure to thrive = wastcd (WHZ < −2 or MUAC < 12.5) or underweight (WHZ < −2) at enrollment (growth trajectories unavailable before enrollment)
^{d} Sputum or gastric aspirate
^{e} Includes 2 children with unconfirmed TB with indeterminate Stool Xpert results
^{f} Includes 1 child with unconfirmed TB and 2 children with unlikely TB who have invalid urine LAM results. Color change corresponding to manufacturer reference card grade ≥1 was considered positive at the time of the study.
^{g} Two children with confirmed TB died before initiating TB treatment
^{h} Within 6 the study period.

TABLE 2
Mtb EV diagnostic performance in 147 hospitalized HIV+ children with symptoms consistent with TB*
A. Mtb EV diagnostic performance

| | Confirmed TB ^{a} | Unconfirmed TB ^{a} (N = 69) | | Unlikely TB ^{a} (N = 67) | |
|---|---|---|---|---|---|
| | Microbiologic | TB diagnosis and | No TB diagnosis or | NIH TB | No NIH TB |
| | confirmation | ATT during study | ATT during study | criteria ^{b} | criteria |
| | (N = 11) | (N = 47) | (N = 22) | (N = 55) | (N = 12) |
| Mtb EV+ children: # | 10 | 35 | 15 | 29 | 2 |
| Sensitivity: % (95% CI) | 90.9 (58.7-99.8) | 74.5 (59.7-86.1) | 68.2 (45.1-86.1) | 52.7 (38.9-66.1) | — |
| Specificity: % (95% CI) | — | — | — | 47.2 (33.9-61.0) | 83.3 (50.8-97.0) |
| Deaths-ATT untreated | 100%; 2/2 | — | 45.5%; 10/22 | 18.2%; 10/55 | 0%; 0/12 |
| Deaths-ATT treated | 33.3%; 3/9 | 8.5%; 4/47 | — | — | — |
| Mtb EV level: | 35.1 (7.5, 68.7) | 16.1 (4.9, 31.6) | 8.6 (4.2, 21.0) | 7.0 (1.8, 24.7) | 2.7 (0.6-4.8) |
| Median (IQR) ^{c} | P = 0.0005 | p = 0.002 | P = 0.005 | p = 0.017 | Reference group |

B. Median Mtb EV level at TB diagnosis and after ATT initiation among children with samples at both time points

| | | Pre-ATT initiation ^{d} | Post-ATT initiation ^{e} | |
|---|---|---|---|---|
| | N | Median EV level (IQR) | Median EV level (IQR) | p-value ^{f} |
| All TB cases | 50 | 15.1 (7.5, 37.4) | 4.9 (1.5, 13.4) | <0.0001 |
| Confirmed TB | 8 | 45.3 (11.6, 66.9) | 3.5 (0.7, 9.5) | 0.002 |
| Unconfirmed TB | 42 | 13.8 (4.9, 35.1) | 4.9 (2.1, 13.4) | 0.004 |

N = 147; 137 children with serum analyzed at baseline; 7 at time of Unconfirmed TB diagnosis (6 at 2-weeks 600 and 1 at 4-weeks post-enrollment); and 3 Unlikely TB cases with missing baseline serum who had serum analyzed at 2-weeks post-enrollment.
ATT: anti-TB treatment.
^{a} Based on international consensus clinical case definitions for pediatric TB (Graham et. al 2015); TB diagnosis and ATT during study - children who were prospectively diagnosed during the study period by clinical staff and who received ATT during the study period; No TB diagnosis or ATT during study - children who were not clinically diagnosed and did not receive ATT during the study period; NIH TB criteria - children who had at least one of the two diagnostic criteria required for TB diagnosis by the NIH algorithm.
^{b} NIH criteria described in Supplementary Table 4.
^{c} Wilcoxon rank sum test compared to Unlikely TB with No TB symptoms and CXR610
^{d} At TB diagnosis or <14 days of TB treatment.
^{e} Latest available samples. Median 5.5 months (IQR 3.1-5.7) between TB treatment initiation and post TB treatment initiation
^{f} Paired Wilcoxon signed-rank test.

What is claimed is:

1. A method of detecting the presence of a Mycobacterium tuberculosis (MTB)-specific protein in a bodily fluid sample, comprising the steps of:
a) extracting extracellular vesicles (EVs) in the bodily fluid sample;
b) mixing antibody-conjugated nanoparticles with the EVs in step a), wherein said antibody-conjugated nanoparticles are conjugated with a first antibody against the MTB-specific protein; and
c) detecting the presence of the MTB-specific protein using dark field microscopy.

2. The method of claim 1, wherein said MTB-specific protein is selected from the group consisting of: lipoarabinomannan (LAM), LAM carrier protein LprG and LpqH, Alpha-crystallin (HspX), DnaK, GroEL2, KatG, SodA and GlnA.

3. The method of claim 1, wherein said first antibody is selected from the group consisting of CS-35, CS-40, and A194-01.

4. The method of claim 1, wherein the conjugation between said first antibody and said nanoparticles is an avidin-biotin or streptavidin-biotin conjugation.

5. The method of claim 4, wherein said first antibody is biotin-functionalized and said nanoparticles are avidin- or streptavidin-functionalized.

6. The method of claim 1, wherein the EVs are extracted by using a second antibody, wherein the second antibody is specific to CD9, CD81, CD91, CD63 CD81, PDCD6IP, HSPA8, ACTB, ANXA2, PKM, HSP90AA1, ENO1, ANXAS, HSP90AB1, YWHAZ, YWHAE, LprG, LpgH, HspX, DnaK, GroEL2, KatG, SodA, or GlnA.

7. The method of claim 1, wherein said nanoparticles are gold nanorods (AuNRs), silver (Ag), bismuth oxide (Bi₂O₃), platinum (Pt), gadolinium oxide (Gd₂O₃), or iron oxide (Fe₃O₄) nanoparticles.

8. A system for detecting an MTB-specific protein in a bodily fluid sample, comprising:
a dark field microscope;
b) a sample substrate; and
c) antibody-conjugated nanoparticles, wherein said antibody-conjugated nanoparticles are conjugated with a first antibody that targets the MTB-specific protein;
wherein the sample substrate is coated with a second antibody against an extracellular vesicle-specific (EV-specific) protein.

9. The system of claim 8, wherein the antibody-conjugated nanoparticles are gold, silver (Ag), bismuth oxide (Bi₂O₃), platinum (Pt), gadolinium oxide (Gd₂O₃), or iron oxide (Fe₃O₄) nanoparticles.

10. The system of claim 8, wherein said MTB-specific protein is selected from the group consisting of lipoarabinomannan (LAM), LAM carrier protein LprG, LpqH, Alpha-crystallin (HspX), DnaK, GroEL2, KatG, SodA and GlnA.

11. The system of claim 8, wherein said first antibody is CS-35, CS-45 or A194-01

12. The system of claim 8, wherein said second antibody is specific to CD9, CD81, CD91, CD63 CD81, PDCD6IP, HSPA8, ACTB, ANXA2, PKM, HSP90AA1, ENO1, ANXAS, HSP90AB1, YWHAZ, YWHAE, LprG, LpgH, HspX, DnaK, GroEL2, KatG, SodA, or GlnA.

13. The system of claim 8, wherein the conjugation between said nanoparticles and said first antibody is biotin-avidin or biotin-streptavidin conjugation.

14. The system of claim 8, wherein the nanoparticles are gold nanoparticles.

15. A method of detecting and determining tuberculosis infection status by detecting the presence of a first and a second Mycobacterium tuberculosis (MTB)-specific proteins in a bodily fluid sample, comprising the steps of:
a) extracting extracellular vesicles (EVs) in the bodily fluid sample;
b) mixing antibody-conjugated nanoparticles with the extracted EVs in step a), wherein said antibody-conjugated nanoparticles are conjugated with a first antibody against the first MTB-specific protein and a second antibody against the second MTB-specific protein;
c) detecting the presence of the first and/or the second MTB-specific proteins using dark field microscopy; and
d) determining the tuberculosis infection status based on the presence of the first and the second MTB-specific proteins.

16. The method of claim 15, wherein the first MTB-specific protein is lipoarabinomannan (LAM), and the second MTB-specific protein is Lipoarabinomannan carrier protein (LprG).

17. The method of claim 15, wherein an active TB infection is determined if both the first and the second MTB-specific proteins are present.

18. A method of screening antibodies against an MTB-specific protein, comprising the steps of
a) immobilizing an MTB-specific protein on a substrate;
b) introducing a plurality of first antibody onto the substrate;
c) mixing nanoparticles with the mixture in step (b), wherein the nanoparticles are conjugated with a second antibody and signal-emitting groups, wherein the second antibody targets the heavy chain constant region of the first antibody; and
d) detecting the presence of antibodies against the MTB-specific protein by detecting the signals emitted by the signal-emitting particles on said nanoparticles.

19. The method of claim 15, wherein the MTB-specific protein is selected from the group consisting of: lipoarabinomannan (LAM), LAM carrier protein LprG and LpqH, and Alpha-crystallin (HspX).

20. The method of claim 15, wherein the MTB-specific protein is LAM or LprG.

21. The method of claim 15, wherein the substrate is coated with Concanavalin A (ConA).

22. A method of detecting the presence of a bacterium-specific protein in a bodily fluid sample, comprising the steps of:
a) extracting extracellular vesicles (EVs) in the bodily fluid sample;
b) mixing antibody-conjugated nanoparticles with the EVs, wherein said antibody-conjugated nanoparticles are conjugated with a first antibody specific to said bacterium-specific protein; and
c) detecting the presence of the bacterium-specific protein using dark field microscopy.
